(19) United States
(12) Patent Application Publication
(10) Pub. No.: US 2019/0336114 A1
(43) Pub. Date: Nov. 7, 2019

(54) INTERNAL JUGULAR CAROTID CLOSURE DEVICE

(71) Applicant: Mubin I. Syed, Springfield, OH (US)

(72) Inventor: Mubin I. Syed, Springfield, OH (US)

(21) Appl. No.: 16/405,204

(22) Filed: May 7, 2019

Related U.S. Application Data
(60) Provisional application No. 62/668,074, filed on May 7, 2018.

(51) Int. Cl. A61B 17/00; A61B 17/12

(52) U.S. Cl. CPC A61B 17/0057; A61B 17/12168; A61B 2017/00606; A61B 2017/081; A61B 2017/00004; A61B 2017/00663; A61B 2017/00778; A61B 2017/00641

(57) ABSTRACT

A closure device is provided. The closure device includes a first planar disk delivered to a first vascular vessel and a second planar disk delivered to a second vascular vessel adjacent to the first vascular vessel. The closure device also includes a compression element configured to pull the first planar disk and the second planar disk together. The first planar disk and the second planar disk are disposed in parallel planes and have a common axis.

### CROSS REFERENCE TO RELATED APPLICATIONS

This application claims priority under 35 U.S.C. § 119 to U.S. Provisional Application No. 62/668,074, entitled “INTERNAL JUGULAR CAROTID CLOSURE DEVICE,” filed on May 7, 2018. The contents of that application are hereby incorporated by reference in their entirety.

### FIELD OF THE INVENTION

This invention relates to fistula closure devices, and in particular to a fistula closure device to be used following internal jugular carotid access.

### BACKGROUND

When performing intravenous procedures in elderly patients, access to peripheral veins such as the upper extremity can be difficult. As a result, the intravenous procedure may require access to the internal jugular vein for central venous access. Arterial closure is typically required after direct carotid access for stroke thrombectomy or a neurovascular procedure (when anatomy for the usual femoral approach is prohibitive).

Because the common carotid artery is adjacent to the internal jugular vein, the common carotid artery is subject to incidental puncturing when accessing the central venous. A medical practitioner typically recognizes the incidental puncturing when red arterial blood flows upon extraction of the needle or tube used to traverse the central venous. The incidental puncturing can have serious implications, such as arterio-venous fistula. Addressing the arterio-venous fistula may require open surgical repair.

Alternatively, uncontrolled bleeding from the common carotid artery can cause blood accumulation in the neck region. The blood accumulation can lead to compression and closing of the trachea, thereby creating a medical emergency. Accordingly, the present disclosure is directed towards a new and improved process of closing an access hole in a common carotid artery.

### SUMMARY

The following is a simplified summary of one or more embodiments in order to provide a basic understanding of present technology. This summary is not an extensive overview of all contemplated embodiments of the present technology. It is intended neither to identify key or critical elements of all examples, nor to delineate the scope of any or all aspects of the present technology. Its sole purpose is to present some concepts of one or more examples in a simplified form as a prelude to the more detailed description that is presented later.

A closure device is provided. The closure device includes a first planar disk delivered to a first vascular vessel and a second planar disk delivered to a second vascular vessel. The second vascular vessel is adjacent to the first vascular vessel. The closure device also includes a compression element configured to pull the first planar disk and the second planar disk together. The first planar disk and the second planar disk are disposed in parallel planes to include a common axis.

In some embodiments, the compression element includes a screw-like threaded member. In other embodiments, the compression element includes threaded sutures. In some embodiments, the first vascular vessel can include the internal jugular vein and the second vascular vessel can include the common carotid artery. The internal jugular vein can include a first and second access hole. The common carotid artery can include a third access hole aligned with the second access hole of the internal jugular vein. The compression element can be configured to pull the first planar disk and the second planar disk together to minimize the third access hole in the common carotid artery.

Embodiments of the disclosure are also directed towards an alternative closure device. The alternative closure device can also include a stent with an anterior end and a posterior end. The posterior end of the stent can be configured to be delivered to a first vascular vessel. The anterior end of the stent can be configured to be delivered to a second vascular vessel, adjacent to the first vascular vessel. The posterior end and the anterior end of the stent can be disposed along a common axis. Moreover, the anterior and posterior ends of the stent can be configured to be twisted together. In some embodiments, the stent can be a double horn stent. In some embodiments, the stent can be made up of a bioabsorbable material.

The present disclosure also provides a process for closing a fistula. The process includes providing a catheter that includes a posterior end and an anterior end. The process also includes inserting the posterior end of the catheter through a first vascular vessel and into a second vascular vessel. The posterior end can be inserted through first and second access holes in the first vascular vessel, and a third access hole in the second vascular vessel. The process also includes providing percutaneous access to the catheter, deploying a first planar disk in the second vascular vessel, deploying a second planar disk in the first vascular vessel, and pulling together the first and second planar disks using a compression element.

In some embodiments of the disclosure, the compression element can include a screw-like threaded member. In alternative embodiments of the disclosure, the compression element can include two or more threaded sutures.

The present disclosure also provides an alternative process for closing a fistula. The alternative process can include providing a catheter with a posterior end and an anterior end. The process can also include inserting the posterior end of the catheter through an internal jugular vein and into a common carotid artery. The process can also include providing percutaneous access to the catheter and inserting the posterior end of the catheter through an internal jugular vein and into a common carotid artery by way of a first access hole and a second access hole in the internal jugular vein, and a third access hole in the common carotid artery. The process can also include deploying a posterior end of an acute double-horned stent into the common carotid artery and deploying an anterior end of the acute double-horned stent into the internal jugular vein. The process can also include inserting an upper prong deployment sheath and a lower prong deployment sheath into the internal jugular vein. The upper prong deployment sheath can include a first prong array and the lower prong deployment sheath can include a second prong array. The process can include inserting a central prong deployment sheath through the internal jugular vein and into the common carotid artery, passing through the acute double-horned stent. The central prong deployment sheath can include a third prong array.

The process also includes retracting the upper prong deployment sheath, the lower prong deployment sheath and the central prong deployment sheath to deploy the first prong array, the second prong array and the third prong array. At least one prong array can be deployed in the internal jugular vein and at least one prong array can be deployed in the common carotid artery. The process also includes configuring the first prong array, the second prong array and the third prong array to engage with an associated portion of the acute double-horned stent. The process can also include applying a first twisting force to the at least one prong array deployed in the internal jugular vein with respect to the at least one prong array deployed in the common carotid artery to compress the acute double-horned stent and close the second and third access holes. The process can finally include applying a second twisting force to the posterior and anterior ends of the acute double-horned stent to minimize the third access hole.

### BRIEF DESCRIPTION OF THE DRAWINGS

In order to describe the manner in which the above-recited and other advantages and features of the disclosure can be obtained, a more particular description of the principles described above will be rendered by reference to specific examples illustrated in the appended drawings. These drawings depict only example aspects of the disclosure and are therefore not to be considered as limiting of its scope. These principles are described and explained with additional specificity and detail through the use of the following drawings.

FIG. 1 illustrates a process of passing a catheter through an internal jugular vein to access a common carotid artery, in accordance with an embodiment of the disclosure;

FIG. 2 illustrates a process of retracting the catheter into the internal jugular vein, in accordance with an embodiment of the disclosure;

FIG. 3 illustrates a process of deploying a planar disk in the internal jugular vein, in accordance with an embodiment of the disclosure;

FIG. 4 illustrates a first and second planar disks drawn together using a threaded member, in accordance with an embodiment of the disclosure;

FIG. 5 illustrates a first planar disk deployed into a common carotid artery through the internal jugular vein, in accordance with an embodiment of the disclosure;

FIG. 6 illustrates a process of retracting the catheter into the internal jugular vein, in accordance with an embodiment of the disclosure;

FIG. 7 illustrates a process of deploying a second planar disk, coupled to the threaded sutures, into the internal jugular vein, in accordance with an embodiment of the disclosure;

FIG. 8 illustrates the first and second planar disks of FIG. 7 drawn together using threaded sutures, in accordance with an embodiment of the disclosure;

FIG. 9 illustrates a posterior end of an acute double-horn stent deployed into a common carotid artery through three access holes, in accordance with an embodiment of the disclosure;

FIG. 10 illustrates a process of retracting the posterior end of the catheter of FIG. 9, from the common carotid artery into the internal jugular vein and deploying an anterior end of the acute double-horn stent into the internal jugular vein, in accordance with an embodiment of the disclosure;

FIG. 11 illustrates an upper prong deployment sheath and a lower prong deployment sheath deployed into the internal jugular vein, and a central prong deployment sheath deployed through the internal jugular vein and into the common carotid artery, passing through the double horn stent of FIG. 10, in accordance with an embodiment of the disclosure;

FIG. 12 illustrates deployment of a prong array from the upper prong deployment sheath, the lower prong deployment sheath, and the central prong deployment sheath of FIG. 11, in accordance with an embodiment of the disclosure;

FIG. 13 illustrates positioning of the prong arrays illustrated in FIG. 12, such that each prong array engages an associated portion of the double horn stent, in accordance with an embodiment of the disclosure.

FIG. 14 illustrates the application of a clockwise twisting force to the pair of deployment sheaths, and a simultaneous application of an anti-clockwise twisting force to the central prong deployment sheath, in accordance with an embodiment of the disclosure;

FIG. 15 illustrates the prong deployment sheaths undergoing further twisting, and the double horn stent undergoing further compression along its axis until its length is minimized, in accordance with an embodiment of the disclosure;

FIG. 16 illustrates the prongs being disengaged from the double-horned stent and the prong deployment sheaths and the associated catheters/sheaths being withdrawn from the treatment area, in accordance with an embodiment of the disclosure;

FIG. 17 illustrates a compressed double-horned stent being deployed to achieve closure of a fistula in the common carotid artery and the first access hole, in accordance with an embodiment of the disclosure;

FIG. 18 is a flow chart illustrating a first process of closing a fistula, in accordance with an embodiment of the disclosure; and

FIG. 19 is a flow chart illustrating a second process of closing a fistula, in accordance with an embodiment of the disclosure.

### DETAILED DESCRIPTION

The present disclosure is described with reference to the attached figures, where like reference numerals are used throughout the figures to designate similar or equivalent elements. The figures are not drawn to scale and are provided merely to illustrate embodiments in the instant disclosure. Several aspects of the disclosure are described below with reference to example applications for illustration. It should be understood that numerous specific details, relationships, and methods are set forth to provide a full understanding of the disclosure. One having ordinary skill in the relevant art, however, will readily recognize that the embodiments of the disclosure can be practiced without one or more of the specific details, or with other methods. In other instances, well-known structures or operations are not shown in detail to avoid obscuring the disclosure. The present disclosure is not limited by the illustrated ordering of acts or events, as some acts may occur in different orders and/or concurrently with other acts or events. Furthermore, not all illustrated acts or events are required to implement a methodology in accordance with the present disclosure.

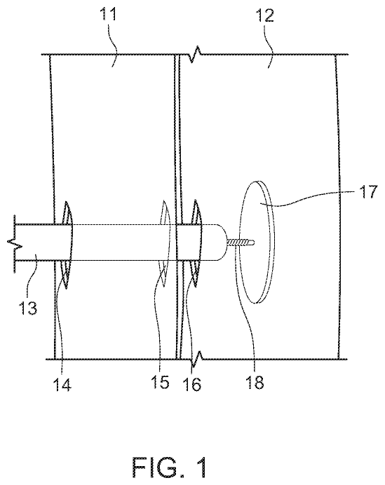

A closure device is provided. The closure device includes a first planar disk delivered to a first vascular vessel and second a planar disk delivered to a second vascular vessel, adjacent to the first vascular vessel. The closure device can also include a compression element configured to pull the first planar disk and the second planar disk together. The first planar disk and the second planar disk can be disposed in parallel planes to include a common axis. FIGS. 1 through 4 are used to describe a first embodiment of the present disclosure. FIG. 1 illustrates a process of passing a catheter 13 through an internal jugular vein 11 to access a common carotid artery 12, in accordance with an embodiment of the disclosure. As illustrated in FIG. 1, a right internal jugular vein 11 is adjacent to a right common carotid artery 12. It should be understood that the embodiments described herein for the right side can be similarly applied to the left side. The right internal jugular vein 11 can include access holes 14 and 15. The common carotid artery 12 can include an access hole 16. The catheter 13 can be configured to penetrate through the access holes 14, 15, and 16, to traverse the right internal jugular vein 11 and enter the right common carotid artery 12. A first planar disk 17 is shown deployed in the right common carotid artery 12. The first planar disk 17 can be made up of memory material, such as Nitinol® (nickel-titanium alloy distinguished from other materials by its shape memory and super elastic characteristics discovered by the Naval Ordnance Laboratory (NOL)). It is understood that Nitinol® is a nickel-titanium alloy distinguished from other materials by its shape memory and super-elastic characteristics. As illustrated herein, the first planar disk 17 can include a planar shape. Other shape memory materials can be implemented as well. The first planar disk 17 can be attached to a screw-like threaded member 18 of the catheter 13. In some embodiments, the first planar disk 17 can be configured in folded forms (not shown) that allow it to be advanced and retracted inside of the catheter 13. For the purposes of this illustration, the access hole 16 can be the result of an inadvertent puncturing of the right common carotid artery 12. Alternatively, the access hole 16 can be surgically created for treatment of a stroke thrombectomy or a neurovascular disorder. It should be understood that access holes 14 and 15 may be necessary to traverse the right internal jugular vein 11 to reach the right common carotid artery 12.

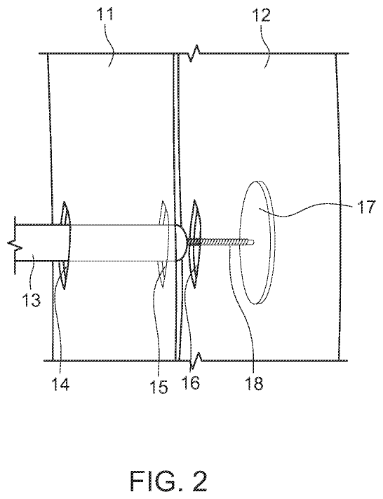

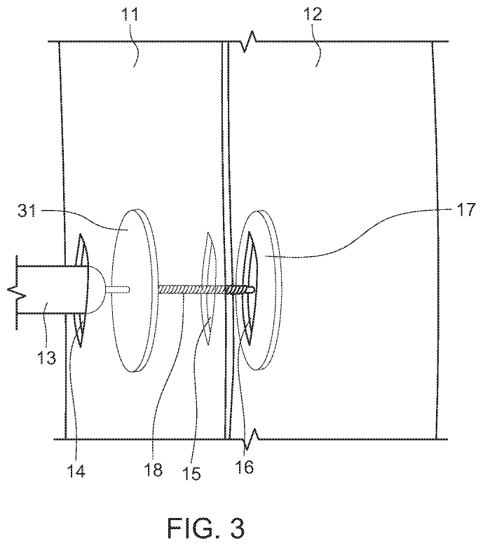

FIG. 2 illustrates a process of retracting the catheter 13 into the internal jugular vein 11, in accordance with an embodiment of the disclosure. The retracting catheter 13 can be retracted into the right internal jugular vein 11 using the screw-like threaded member 18. FIG. 3 illustrates a process of deploying a planar disk in the internal jugular vein, in accordance with an embodiment of the disclosure FIG. 3 shows the deployment of a second planar disk 31 into the right internal jugular vein 11, again using screw-like threaded member 18 to position the second planar disk 31.

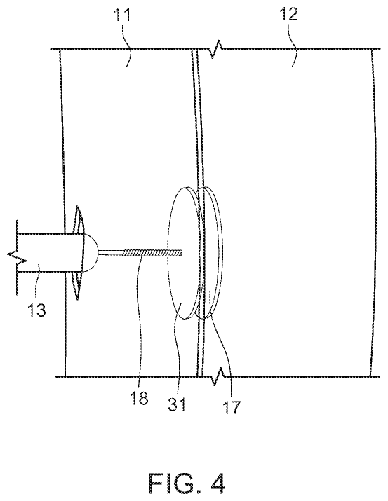

FIG. 4 illustrates a first planar disk and second planar disks 31 drawn together using the threaded member 18, in accordance with an embodiment of the disclosure. For the purposes of this illustration, the adjacent walls of the right common carotid artery 12 and the right internal jugular vein 11 can be compressed together. This pressing together effectively seals the third access hole 16 (not shown herein due to the pair of planar disks). The screw-like threaded member 18 can include an intravenous portion that may be made of a biodegradable, blood soluble polymer. Other portions of the screw-like threaded member 18 can be made of non-soluble (permanent) material; for example, the tip and a portion between the planar disks. The screw-like threaded member 18 serves to compress the posterior wall of the internal jugular vein 11 against the adjacent anterior wall of the common carotid artery 12. The threaded and biodegradable portion of the screw-like threaded member 18 can be severed inside the vein close to the planar disk by gentle traction, twisting or angulation.

The process of sealing the access hole in the right common carotid artery is expeditious and innocuous. Direct access can be achieved using the neck region and internal jugular routing. As a result, the process of sealing the access hole in the right common carotid artery is quick. Speed is an important element in this procedure due to the catastrophic result of blood build up in the neck region. As indicated above, an accumulation of blood can apply pressure to the trachea and block the flow of air, thereby creating a medical emergency. The walls of the right common carotid artery and the right internal jugular vein are well supported near the third access hole, thereby creating an effective seal.

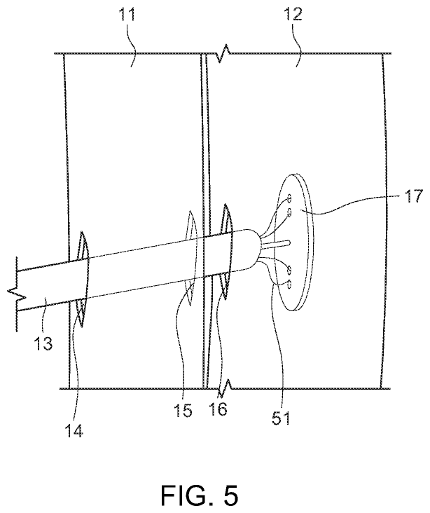

FIGS. 5 through 8 are used to describe a second embodiment of the present disclosure, wherein the pair of planar disks are drawn in together using threaded sutures. FIG. 5 illustrates a first planar disk 17 deployed into a common carotid artery 12 through the internal jugular vein 11, in accordance with an embodiment of the disclosure. The first planar disk 17 can be deployed through the catheter 13 into the right common carotid artery 12. The first planar disk 17 can include apertures configured to receive threaded sutures 51. The threaded sutures 51 can be attached to the first planar disk 17 and to the catheter 13. First, second and third access holes 14, 15 and 16 are shown herein. The access holes are similar to those discussed above with respect to FIG. 1.

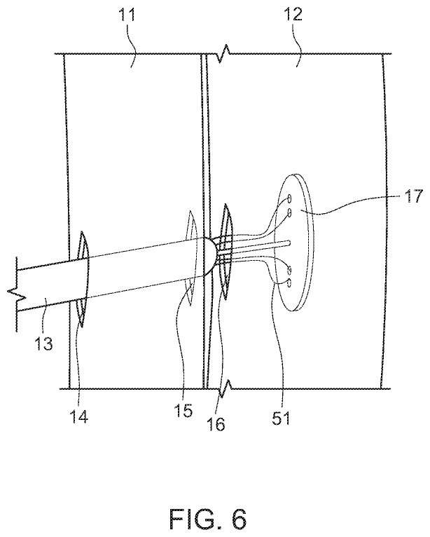

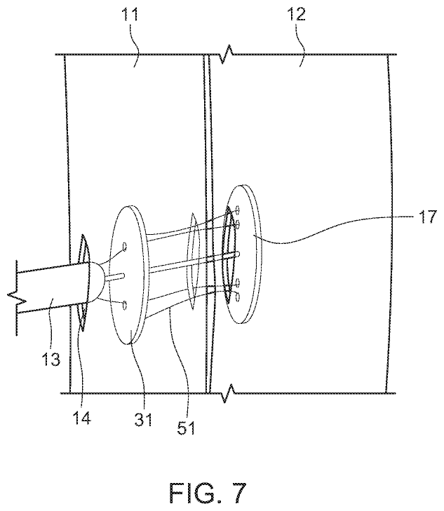

FIG. 6 illustrates a process of retracting the catheter 13 into the internal jugular vein 11, in accordance with an embodiment of the disclosure. FIG. 7 illustrates a process of deploying a second planar disk 31, coupled to the threaded sutures 51, into the right internal jugular vein 11, in accordance with an embodiment of the disclosure.

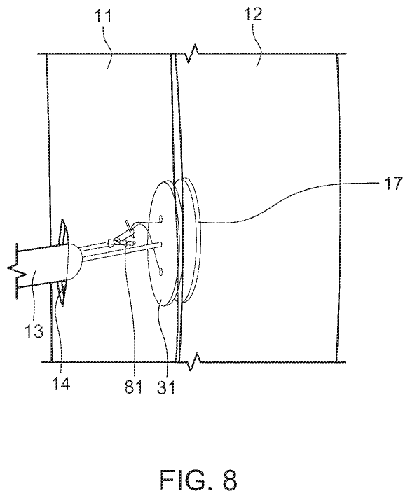

FIG. 8 illustrates the first planar disk 17 and second planar disk 31 drawn together using the threaded sutures 51, in accordance with an embodiment of the disclosure. A knot tying device 81 can be implemented herein to secure the threaded sutures 51 after the first planar disk 17 and second planar disk 31 are pressed together. The third access hole 16 is effectively sealed by the pairing of the first planar disk 17 and second planar disk 31. As discussed above, the process of sealing the access hole in the right common carotid artery 12 is expeditious and innocuous.

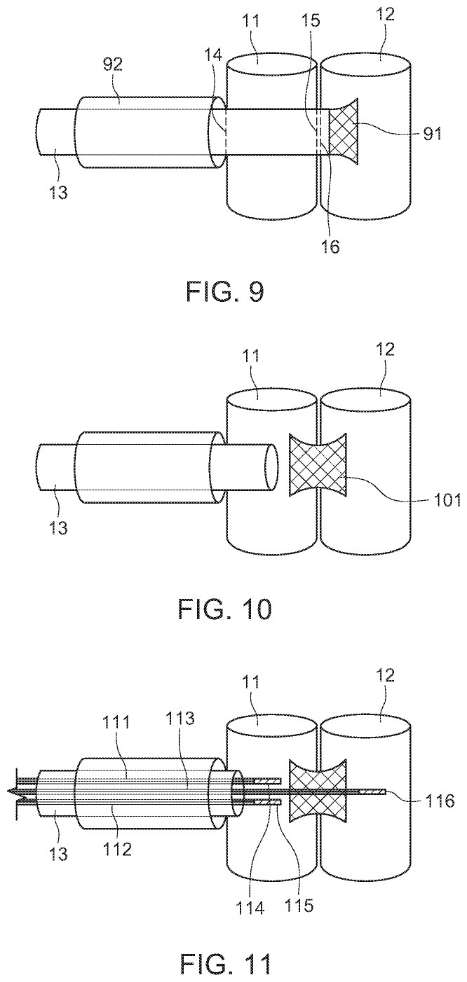

FIG. 9 illustrates a posterior end 91 of an acute double-horn stent deployed into a common carotid artery 12, in accordance with an embodiment of the disclosure. The posterior end 91 of the acute double-horn stent is deployed into the right common carotid artery 12 using the catheter 13 disposed inside a sheath 92. The catheter 13 is configured to traverse the right internal jugular vein 11 through the first, second, and third access holes 14, 15, and 16. The stent can be made up of bioabsorbable materials.

FIG. 10 illustrates a process of retracting the posterior end 91 of the catheter 13 of FIG. 9, from the right common carotid artery 12 into the right internal jugular vein 11. FIG. 10 also illustrates an anterior end 101 of the acute double-horned stent deployed in the right jugular vein, in accordance with an embodiment of the disclosure.

FIG. 11 illustrates an upper prong deployment sheath 111, a lower prong deployment sheath 112, and a central prong deployment sheath 113. The upper prong deployment sheath 111 and the lower prong deployment sheath 112 can be threaded into the right internal jugular vein 11. The central prong deployment sheath 113 can be threaded through the right internal jugular vein 11 and into the common carotid artery 12, passing through the acute double-horned stent. In some embodiments, the upper prong deployment sheath 111 can include a folded array of prongs 114. Likewise, the lower prong deployment sheath 112 can include a folded array of prongs 115. The central prong deployment sheath 113 can also include a folded array of prongs 116.

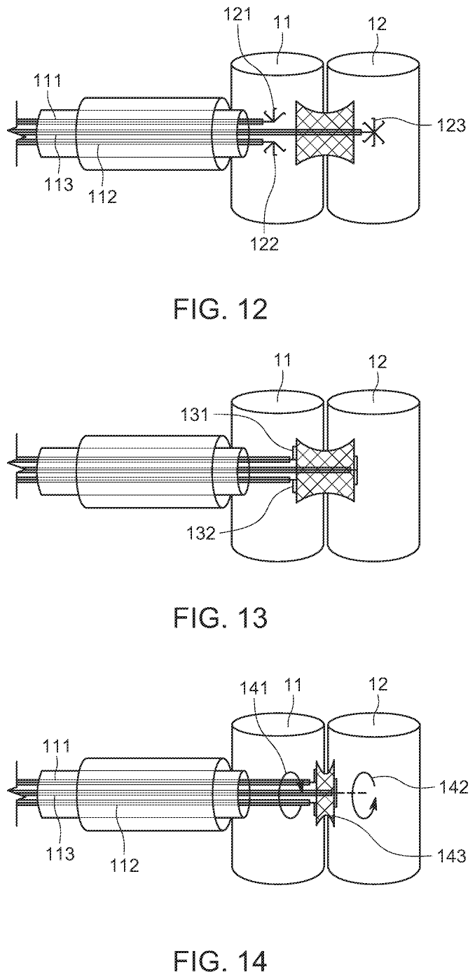

FIG. 12 illustrates the upper prong deployment sheath 111 being retracted and the prong array 121 being deployed. Similarly, the lower prong deployment sheath 112 is retracted and the prong array 122 is deployed. The prong deployment sheath 113 is also retracted and the prong array 123 is also deployed.

FIG. 13 illustrates an engagement 131 of the prong array with the anterior end of the acute double-horned stent. The prong array 122 is also engaged 132 with the anterior end of the acute double-horned stent. Finally, the prong array 123 is engaged 133 with the posterior end of the acute double-horned stent.

FIG. 14 illustrates an application of a clockwise twisting force 141 to the upper and lower deployment sheaths 111, 112. Simultaneously, an anti-clockwise twisting force 142 can be applied to the central prong deployment sheath 113. The anti-clockwise twisting force 142 serves to compress 143 the acute double-horned stent along its axis (not shown) to provide a seal over the third access hole (not shown) in the right common carotid artery 12. In some embodiments, the application of the anti-clockwise twisting force 142 causes the acute double-horned stent to conform to the shape of a hyperboloid, as shown herein. As a result, the process of sealing the access hole in the right common carotid artery 12 is expeditious and innocuous.

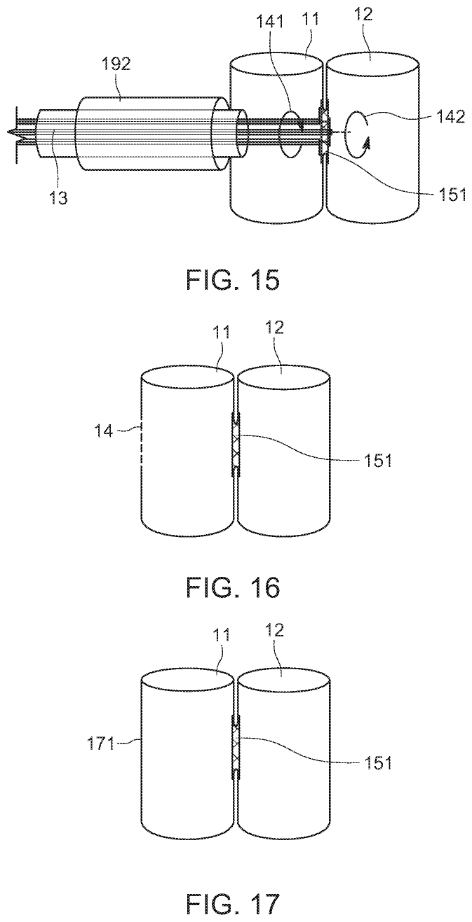

FIG. 15 illustrates an application of a compression force 151 of the acute double-horned stent as a result of the clockwise twisting force 141 and the anti-clockwise twisting force 142, discussed above with respect to FIG. 14. FIG. 16 illustrates the compressed double horn stent 151, which has pulled together adjacent walls of the interior jugular vein 11 and the common carotid artery 12 at the location of the second access hole (not shown). Tension in the wires (not shown) of the acute double-horned stent will cause the third access hole (not shown) to at least substantially close, and preferably to fully close. Thus, the second and third access holes and the adjacent fistula are effectively sealed by the twisted stent configuration. It should be understood that each prong array is disengaged from the acute double-horned stent by reversing the twisting forces discussed above, and retracting each prong array into its associated prong deployment catheter. The catheter, the upper prong deployment sheath, the lower prong deployment sheath, and the central prong deployment sheath are all retracted. Furthermore, the folded prongs inside of each prong deployment sheath is also retraced. Retraction of the sheath has left the first access hole 14 open.

FIG. 17 illustrates the first access hole 14 closed. Closure of the first access hole 14 can be achieved using known gentle manual compression techniques.

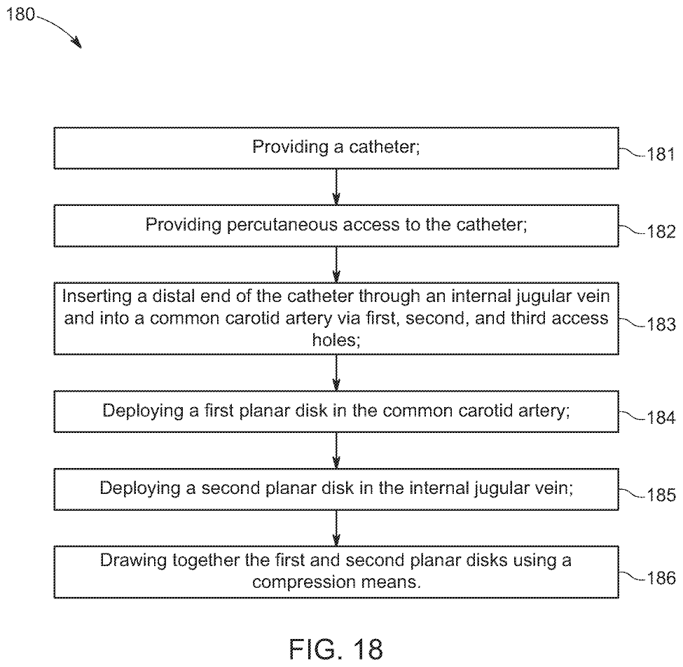

FIG. 18 is a flow chart illustrating a first process 180 of closing a fistula, in accordance with an embodiment of the disclosure. The process 180 includes providing a catheter at step 181. At step 182, a percutaneous access is provided to the catheter. The process advances to step 183, where a posterior end of the catheter is inserted through an internal jugular vein and into a common carotid artery. As discussed above, the catheter is inserted into the common carotid artery via first, second, and third access holes. The process 18 advances to step 184, where a first planar disk is deployed in the common carotid artery. The process 180 advances to step 185 where a second planar disk is deployed in the internal jugular vein. The process 180 advances to step 186 where the first and second planar disks are drawn together using a compression element. As discussed above with respect to the various embodiments disclosed herein, the compression element can include a screw-like threaded member or a threaded suture. The process 180 concludes after step 186.

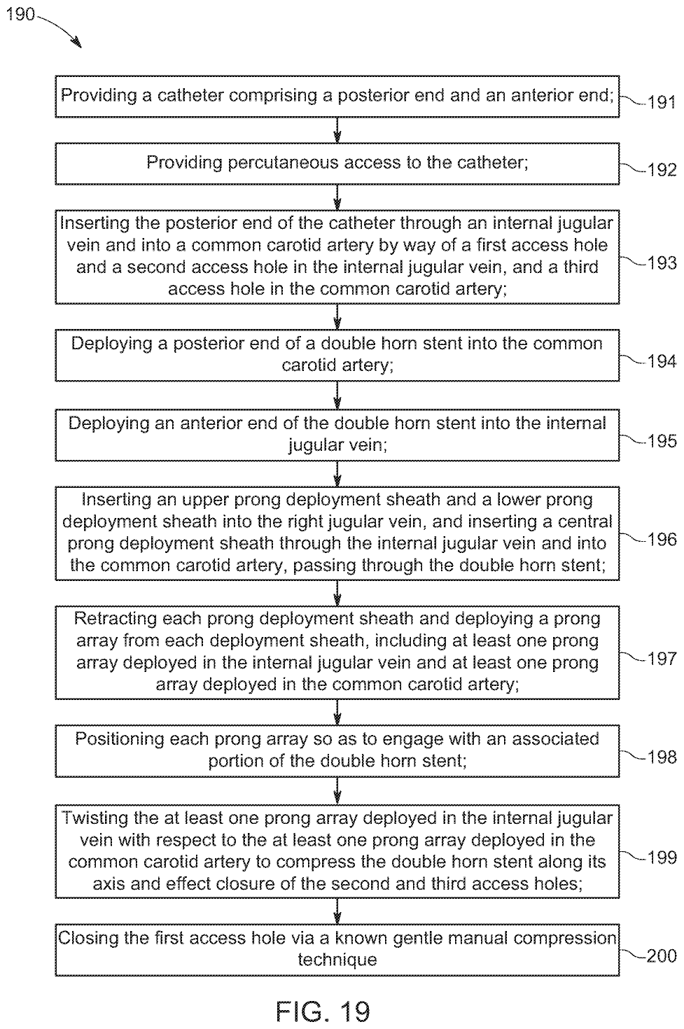

FIG. 19 is a flow chart of an alternative process 190 for closing a fistula using Internal Jugular carotid access. The process 190 initiates at step 191, where a catheter, which includes a posterior end and an anterior end, is provided. The process 190 advances to step 192 where percutaneous access is provided to the catheter. At step 193 a posterior end of the catheter is inserted through the internal jugular vein and into a common carotid artery through first, second, and third access holes. The process 190 advances to step 194 where a posterior end of an acute double-horned stent is deployed into the common carotid artery. At step 195 an anterior end of the double horn stent is deployed into the internal jugular vein. The process 190 advances to step 196 where an upper prong deployment sheath and a lower prong deployment sheath are inserted into the right jugular vein and a central prong deployment sheath is inserted through the internal jugular vein and into the common carotid artery, passing through the double horn stent.

At step 197 the upper prong deployment sheath is retracted to deploy a first prong array, the lower prong deployment sheath is retracted to deploy a second prong array, and the central prong deployment sheath is retracted to deploy a third prong array. The process 190 advances to step 198 where the first and second prong arrays are engaged in the anterior end of the acute double-horned stent. The third prong array is also engaged in the posterior end of the acute double-horned stent. At step 199 the first and second prong arrays are twisted clockwise, while the third prong array is simultaneously twisted counter-clockwise to compress the acute double-horned stent along its axis until the second and third access holes are closed. The process advances to step 200 where each prong array is disengaged from the acute double-horned stent and all sheaths and catheters are retracted from the treatment area. The folded prongs inside of each prong deployment sheath is also retracted. The process 190 concludes at step 201, where the first access hole is closed via a known gentle manual compression technique.

Embodiments of the present disclosure, including the processes described in FIG. 18 and FIG. 19, can be implemented anywhere in the body where arteries and/or veins are juxtaposed and there is a lumen between them. Non-limiting examples of such vascular vessel constructions include, for example, trans popliteal vein to popliteal artery access/fistulas, trans subclavian vein to subclavian artery access/fistulas, and transfemoral artery to femoral vein access/fistulas. Accordingly, the processes described herein can be applied anywhere there is a first vascular vessel adjacent a second vascular vessel and an access hole or fistula between them.

While particular embodiments of the present invention have been shown and described, it will be obvious to those skilled in the relevant arts that changes and modifications may be made without departing from the invention in its broader aspects. Therefore, the aim in the appended claims is to cover all such changes and modifications that fall within the true spirit and scope of the invention. The matter set forth in the foregoing description and accompanying drawings is offered by way of illustration only and not as a limitation. The actual scope of the invention is intended to be defined in the following claims when viewed in their proper perspective based on the prior art.

The terminology used herein is for the purpose of describing particular embodiments only and is not intended to limit the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

What is claimed is:

1. A closure device comprising:
a first planar disk configured to be delivered to a first vascular vessel using a catheter;
a second planar disk configured to be delivered to a second vascular vessel using the catheter, wherein the second vascular vessel is adjacent to the first vascular vessel; and
a compression element configured to pull the first planar disk and the second planar disk together,
wherein the first planar disk and the second planar disk are disposed in parallel planes and have a common axis.

2. The closure device of claim 1, wherein the compression element comprises a screw-like threaded member.

3. The closure device of claim 1, wherein the compression element comprises two or more threaded sutures.

4. The closure device of claim 1, wherein the first vascular vessel is an internal jugular vein and the second vascular vessel is a common carotid artery.

5. The closure device of claim 4, wherein the internal jugular vein comprises a first and second access hole, and the common carotid artery comprises a third access hole aligned with the second access hole of the internal jugular vein.

6. The closure device of claim 5, wherein the compression element is configured to pull the first planar disk and the second planar disk together to close the third access hole in the common carotid artery.

7. A closure device comprising:
a stent comprising an anterior end and a posterior end;
wherein the posterior end of the stent is configured to be delivered to a first vascular vessel and the anterior end of the stent is configured to be delivered to a second vascular vessel adjacent the first vascular vessel;
wherein the posterior end and the anterior end of the stent are disposed along a common axis and the anterior and posterior ends of the stent are twisted together.

8. The closure device of claim 7, wherein the stent comprises a double horn stent.

9. The closure device of claim 7, wherein the stent comprises a bioabsorbable material.

10. The closure device of claim 7, wherein the first vascular vessel is a common carotid artery and the second vascular vessel is an internal jugular vein.

11. The closure device of claim 10, wherein the internal jugular vein comprises a first and second access hole, and the common carotid artery comprises a third access hole aligned with the second access hole of the internal jugular vein.

12. The closure device of claim 11, wherein the anterior and posterior ends of the stent are twisted together to close the third access hole in the common carotid artery.

13. The closure device of claim 11, wherein the posterior end of the stent is configured to be delivered to the common carotid artery by way of the first access hole, the second access hole and the third access hole.

14. The closure device of claim 11, wherein the anterior end of the stent is configured to be delivered to internal jugular vein by way of the first access hole.

15. A method for closing a fistula comprising:
providing a catheter comprising a posterior end and an anterior end;
inserting the posterior end of the catheter through a first vascular vessel and into a second vascular vessel by way of a first access hole and a second access hole in the first vascular vessel, and a third access hole in the second vascular vessel;
providing percutaneous access to the catheter;
deploying a first planar disk in the second vascular vessel;
deploying a second planar disk in the first vascular vessel; and
pulling together the first and second planar disks using a compression element.

16. The method of claim 15, wherein the compression element comprises a screw-like threaded member.

17. The method of claim 15, wherein the compression element comprises two or more threaded sutures.

18. The method of claim 15, wherein the first vascular vessel is an internal jugular vein and the second vascular vessel is a common carotid artery.

19. The method of claim 15, wherein pulling together the first and second planar disks using the compression element minimizes the third access hole in the second vascular vessel.

20. A method for closing a fistula comprising:
providing a catheter comprising a posterior end and an anterior end;
providing percutaneous access to the catheter and inserting the posterior end of the catheter through an internal jugular vein and into a common carotid artery by way of a first access hole and a second access hole in the internal jugular vein, and a third access hole in the common carotid artery;
deploying a posterior end of an acute double-horned stent into the common carotid artery;
deploying an anterior end of the acute double-horned stent into the internal jugular vein;
inserting an upper prong deployment sheath and a lower prong deployment sheath into the internal jugular vein, wherein the upper prong deployment sheath comprises a first prong array and the lower prong deployment sheath comprises a second prong array;
inserting a central prong deployment sheath through the internal jugular vein and into the common carotid artery, passing through the acute double-horned stent, wherein the central prong deployment sheath comprises a third prong array;
retracting the upper prong deployment sheath, the lower prong deployment sheath and the central prong deployment sheath to deploy the first prong array, the second prong array and the third prong array, wherein at least one prong array is deployed in the internal jugular vein and at least one prong array is deployed in the common carotid artery;
configuring the first prong array, the second prong array and the third prong array to engage with an associated portion of the acute double-horned stent;
applying a first twisting force to the at least one prong array deployed in the internal jugular vein with respect to the at least one prong array deployed in the common carotid artery to compress the acute double-horned stent and close the second and third access holes; and
applying a second twisting force to the posterior and anterior ends of the acute double-horned stent to minimize the third access hole.
